(19) United States
(12) Patent Application Publication
(10) Pub. No.: US 2015/0231417 A1
(43) Pub. Date: Aug. 20, 2015

(54) SPINAL SURGERY SYSTEM AND METHOD

(71) Applicant: Warsaw Orthopedic, Inc., Warsaw, IN (US)

(72) Inventors: Newton H. Metcalf, Memphis, TN (US); Thomas A. Carls, Memphis, TN (US); Kevin T. Foley, Germantown, TN (US)

(73) Assignee: Warsaw Orthopedic, Inc., Warsaw, IN (US)

(21) Appl. No.: 14/185,308

(22) Filed: Feb. 20, 2014

(51) Int. Cl. A61N 7/02

(52) U.S. Cl. CPC A61N 7/02

(57) ABSTRACT

A method for treating a spine comprising the steps of: providing a magnetic resonance imaging (MRI) device; identifying a surgical site for treatment of a spinal disorder with the MRI device, the surgical site including a portion of a spine; providing a high intensity focused ultrasound (HIFU) device including a transducer for emitting ultrasound energy; determining parameters of treatment for the surgical site; and applying a dosage of ultrasound energy to the surgical site with the HIFU device for treating the disorder. Systems and devices are disclosed.

### TECHNICAL FIELD

The present disclosure generally relates to medical devices for the treatment of musculoskeletal disorders, and more particularly to a spinal surgery system for treating pathologies of the spine and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics.

Magnetic-resonance-guided high intensity focused ultrasound. (MRgHIFUS) utilizes real-time magnetic resonance guidance to direct high intensity focused ultrasound beams to ablate tissues within the body. MRgHIFUS provides a non-invasive and non-irradiating method of ablating tissue. High intensity focused ultrasound (HIFU) generates focused ultrasonic beams, which converge a distance away from the point of origin of the ultrasonic beams to cause, for example, ablation and/or coagulation necrosis of tissue by over-stimulation of the tissue as sonic energy is converted into thermal energy. The individual ultrasonic beams can travel within various tissues while having minimal to no effect on the tissues it travels through. This disclosure describes an improvement over these prior art technologies.

### SUMMARY

In one embodiment, a method for treating a spine is provided. The method comprising the steps of: providing a magnetic resonance imaging (MRI) device; identifying a surgical site for treatment of a spinal disorder with the MRI device, the surgical site including a portion of a spine; providing a high intensity focused ultrasound (HIFU) device including a transducer for emitting ultrasound energy; determining parameters of treatment for the surgical site; and applying a dosage of ultrasound energy b the surgical site with the HIFU device for treating the disorder. In some embodiments, systems and devices are disclosed.

### BRIEF DESCRIPTION OF THE DRAWINGS

The present disclosure will become more readily apparent from the specific description accompanied by the following drawings, in which:

FIG. 1 is a plan view of components of one embodiment of a surgical system in accordance with the principles of the present disclosure disposed with a body;

FIG. 2 is a cutaway perspective view of the components and body shown in FIG. 1;

FIG. 3 is a perspective view in part crass section of the components and vertebrae of the body shown in FIG. 1;

FIG. 4 is a side view of components of the system shown in FIG.

FIG. 5 is a side view of components of the system shown in FIG. 1; and

FIG. 6 is a flow chart of a method of one embodiment of a surgical system in accordance with the principles of the present disclosure.

### DETAILED DESCRIPTION

The exemplary embodiments of a surgical system are discussed in terms of medical devices for the treatment of musculoskeletal disorders and more particularly to a spinal surgery system for treating pathologies of the spine and a method for treating a spine.

In some embodiments, the present disclosure provides a surgical system that employs focused ultrasound, which comprises a combination of a high intensity focused ultrasound (HIFU) device and a magnetic resonance imaging (MRI) device. In some embodiments, the MRI device identifies target tissue of a spine to be treated and guides the treatment interactively in real time, providing immediate confirmation of the effectiveness of a spinal treatment therapy. In some embodiments, the HIFU device emits a focused ultrasound that concentrates intersecting beams of ultrasound energy with precision on a spinal tissue target deep in a body by focusing the beams on a single point of the spine. In some embodiments, the HIFU device focuses each individual beam of focused ultrasound and passes the individual beam through intermediate tissue, which the beams have no effect, and the beams converge on the target tissue to ablate target tissue.

In one embodiment, the system is employed with a method that utilizes MRgHIFUS for focused spinal tissue ablation. In some embodiments, the method includes a focal spot that is the size of a grain of rice. In one embodiment, the method includes a focal point of the HIFU device that is approximately 5 millimeters (mm). Tissue not contacted by the focal point of the HIFU device is not damaged. In one embodiment, the focal point is accurate to within 0.5 mm. In some embodiments, the method provides for a real-time assessment.

In one embodiment, the system is employed with a method that employs HIFU for targeting spinal tissue. In one embodiment, the system is employed with a method for treatment of a herniated nucleus pulposus. In one embodiment, the system is employed with a method for treatment of spinal tumors. In one embodiment, the system is employed with a method for ablation of hypertrophic ligamentum flavum. In one embodiment, the system is employed with a method for ablation of a basivertebral nerve and facet rhizotomy. In one embodiment, a method for localizing and treating spinal pathologies using HIFU is provided. In one embodiment, the system is employed with a method for performing a non-invasive and non-irradiation spinal surgery, such as, for example, focal decompression, capable of precisely treating spinal pathologies.

In some embodiments, the present disclosure provides a surgical system that employs MRgHIFU for the spine and specific algorithms to account and/or navigate the complex bony anatomy of the spine. In one embodiment, the system is employed with a method that protects the sensitive neural structures of the spine from heat produced by the HIFU device. In one embodiment, the system is employed with a method that includes algorithms, such as, for example, for pulsing energy to control heat profusion. In one embodiment, the system includes a heat sensor. In one embodiment, the algorithms are provided to precisely treat the anatomical structures, such as, for example, discs and nerves, which have different biochemical compositions.

In some embodiments, one or all of the components of the system may be disposable, peel pack and/or pre packed sterile devices. One or all of the components of the system may be reusable. The system may be configured as a kit with multiple sized and configured components.

In some embodiments, the system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the system of the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The system of the present disclosure may also be alternatively employed with procedures for treating the lumbar, cervical, thoracic, sacral and pelvic regions of a spinal column. The system of the present disclosure may also be used on animals, bone models and other non-living substrates, such as, for example, in training, testing and demonstration.

The system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, treating or treatment of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term tissue includes soft tissue, ligaments, tendons, cartilage and/or bone unless specifically referred to otherwise.

In some embodiments, the terms therapeutic transducer, HIFU transducer, and high intensity transducer, as used herein and in the claims that follow, all refer to a transducer that is capable of being energized to produce ultrasonic waves that are more energetic than the ultrasonic pulses produced by an imaging transducer, and which can be focused or directed onto a discrete location, such as a treatment site in a target area

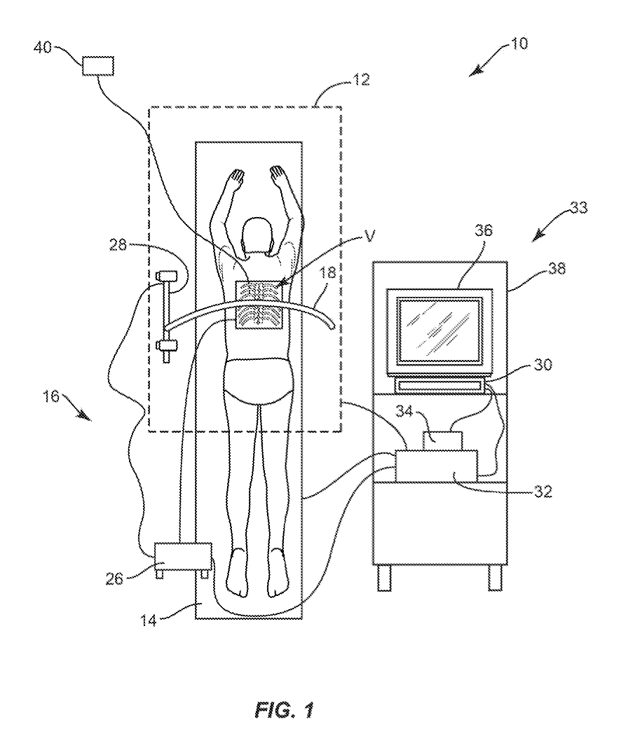

The following discussion includes a description of a surgical system including medical devices, related components and methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-6, there are illustrated components of a system, such as, for example, a MRgHIFU 10 for treating a spine, in accordance with the principles of the present disclosure.

System 10 includes an MRI device 12 for identifying a portion of a spine requiring treatment. MRI device 12 includes a body, such as, for example, a cylindrical body 15 defining a bore 17 configured for disposal of a patient. In some embodiments, MRI device 12 includes a magnet capable of producing a magnetic field of about 0.5 to greater than 3.0 tesla. MRI device 12 includes a radio frequency coil, a gradient coil, a scanner and a patient table 14.

When a patient is positioned within bore 17, a strong magnetic field emitted by the magnet causes atoms, such as, for example, hydrogen atoms to line up in a specific orientation relative to the magnetic field. A radio frequency pulse is applied to the patient by the radio frequency coil, which excites hydrogen atoms in the patient. The gradient coils are manipulated in a manner to alter the magnetic fields at a local level to enable the taking of an imaging slice or a three-dimensional image of a portion of the body. The radio frequency signal is turned off causing the hydrogen atoms to send radio frequency signals, which are picked up by MRI device 12, and are converted into an image.

MRI device 12 can be used to image various tissues and anatomy of the body, such as, for example, vertebrae V, as shown in FIG. 1. MRI device 12 is capable of providing three-dimensional images of the region in vertebrae V that requires treatment in real-time. The real-time imaging of vertebrae V provides real-time monitoring of the procedure such that the practitioner has constant visual feedback of the procedure being performed. MRI device 12 is configured to provide a temperature image, providing a practitioner with information on the relative temperatures of the tissues being operated on.

System 10 includes a HIFU device 16 having an instrument including an ultrasound transducer, such as, for example, a high intensity transducer 18, and a phase array generator 26. The instrument including transducer 18 is configured to treat the portion of the spine requiring treatment. In one embodiment, transducer 18 is a phased array ultrasound transducer including approximately 256 elements or channels capable of being pulsed independently, in a predefined sequence. Transducer 18 includes a surface 20 having a concave configuration that emits approximately 1,000 ultrasonic beams at focal intensities of about 1000-10000 W/cm² for surgical applications for treating spinal tissue. In some embodiments, transducer 18 emits a focal intensity of about 1480-1850 W/cm² and/or about 1500-1930 W/cm² for surgical applications for treating spinal tissue. Transducer 18 is configured to emit ultrasound beams 22 from each element at a plurality of angles with respect to surface 20. In some embodiments, all or only a portion of surface 20 has alternate surface configurations, such as, for example, parabolic, tubular, oval, oblong, triangular, square, polygonal, irregular, uniform, non-uniform, variable, undulating, and/or tapered. In some embodiments, the instrument includes one or more probes configured for disposal of transducer 18 and/or imaging transducers.

Surface 20 focuses ultrasonic beams 22 such that beams 22 converge at a focal point 24, as shown in FIG. 3, a distance away from transducer 18. In some embodiments, transducer 18 has a focal length from about 25 μm to about 3500 μm, a focal distance from about 80 mm to about 200 mm, a diameter from about 80 mm to about 300 mm, a working frequency of about 0.5 MHz to about 1 GHz and an amplitude from about 1 micron to about 100 microns, for surgical applications for treating spinal tissue.

In some embodiments, the focal length is about 3500 μm, 3250 μm, 3000 μm, 2750 μm, 2500 μm, 2250 μm, 2000 μm, 1750 μm, 1500 μm, 1250 μm, 1000, μm 750 μm, 500 μm, 250 μm, about 175 μm, 150 μm, 125 μm, 100 μm, 75 μm, 50 μm and 25 μm for surgical applications for treating spinal tissue. In some embodiments, the focal length is about 140 μm, 70 μm, 50 μm and 35 μm. In some embodiments, the focal distance is about 80 mm, 90 mm, 100 mm, 110 mm, 120 mm, 130 mm, 140 mm, 150 mm, 160 mm, 170 mm, 180 mm, 190 mm and 200 mm for surgical applications for treating spinal tissue. In some embodiments, the diameter is about 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, 290 to about 300 mm for surgical applications for treating spinal tissue. In some embodiments, the working frequency range for transducer 18 is from about 20 kHz to about 100 kHz, from about 25 kHz to about 50 kHz, or from about 30 kHz to about 50 kHz for surgical applications for treating spinal tissue. In some embodiments, the working frequency range for transducer 18 is from about 0.7 MHz to about 3 MHz, from about 0.5 MHz to about 1.0 MHz, from about 0.7 MHz to about 1.0 MHz, from about 1.0 MHz to about 10.0 MHz, or from about 5.0 MHz to about 10.0 MHz for surgical applications for treating spinal tissue. In some embodiments, the working frequency of transducer 18 is from about 0.5 MHz to about 2 MHz, from about 100 MHz to about 400 MHz and from about 500 MHz to about 1 GHz for surgical applications for treating spinal tissue. In some embodiments, the amplitude is from about 1 micron to about 5 microns, from about 5 microns to about 10 microns, from about 5 microns to about 30 microns, from about 30 microns to about 100 microns for surgical applications for treating spinal tissue. In one embodiment, these parameters may include the type of tissue being treated, the age of the patient, the condition of the spine and the size of a spinal defect or location.

In some embodiments, transducer 18 has an acoustic output of 60 W at a duty cycle of 55%, and an acoustic intensity of 1480-1850 W/cm² (ISATA, spatial-average, temporal-average) for surgical applications for treating spinal tissue. In some embodiments, transducer 18 operates at a scanning rate of HIFU application 0.5-0.6 mm/s for surgical applications for treating spinal tissue. In one embodiment, temperature at a face of transducer 18 with HIFU of 60 W acoustic power at 55% duty cycle and duration of 40 seconds, increases from a baseline of 21.3° C. to 35.9° C. for surgical applications for treating spinal tissue. In some embodiments, temperature returns to a baseline 10 minutes after treatment.

Phase array generator 26 is coupled to transducer 18 providing energy to activate transducer 18. Generator 26 can independently set the frequency, phase and amplitude of each channel of transducer 18. Generator 26 can generate up to 3 watts per channel and has a switching time of less than 1 millisecond.

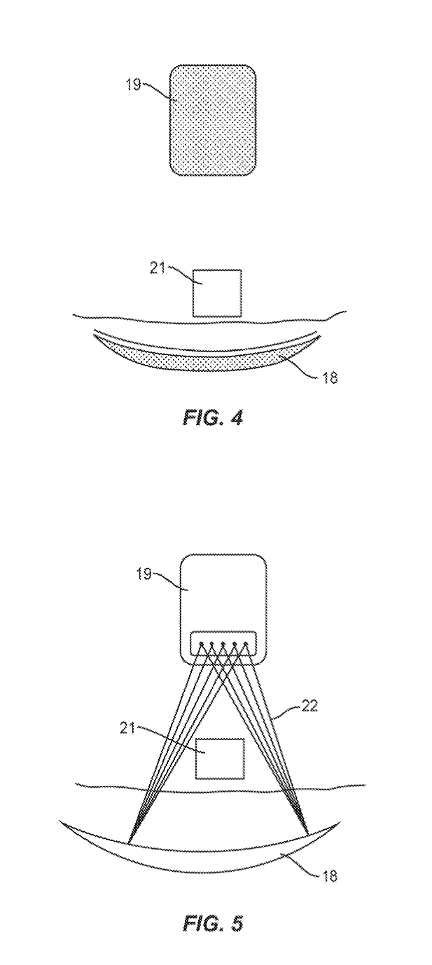

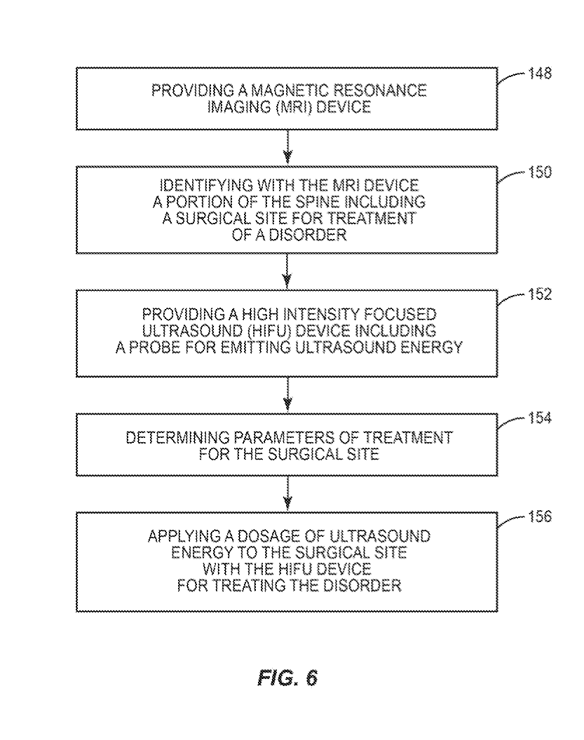

System 10 includes a mechanical positioning device 28 configured to hold and position transducer 18. Device 28 is electronically coupled to a computer 30 via a processor 32 that controls the position of transducer 18 relative to the targeted site to be treated. Processor 32 executes computer-executable instructions for optimizing transducer 18, the instructions comprising evaluating transducer 18 data including transducer 18 position, geometry, and acoustic parameter information. The instructions further include evaluating 3D MR data including region of interest 19 to be ablated data describing a size, shape, and position of region of interest 19 to be ablated, and obstruction data describing a size, shape, and position of an obstruction between one or more transducer 18 elements and region of interest 19, as shown in FIGS. 4 and 5. Device 28 is configured to move transducer 18 in the sagittal and transverse planes in fine steps such that focal point 24 of HIFU device 16 precisely targets the tissue site.

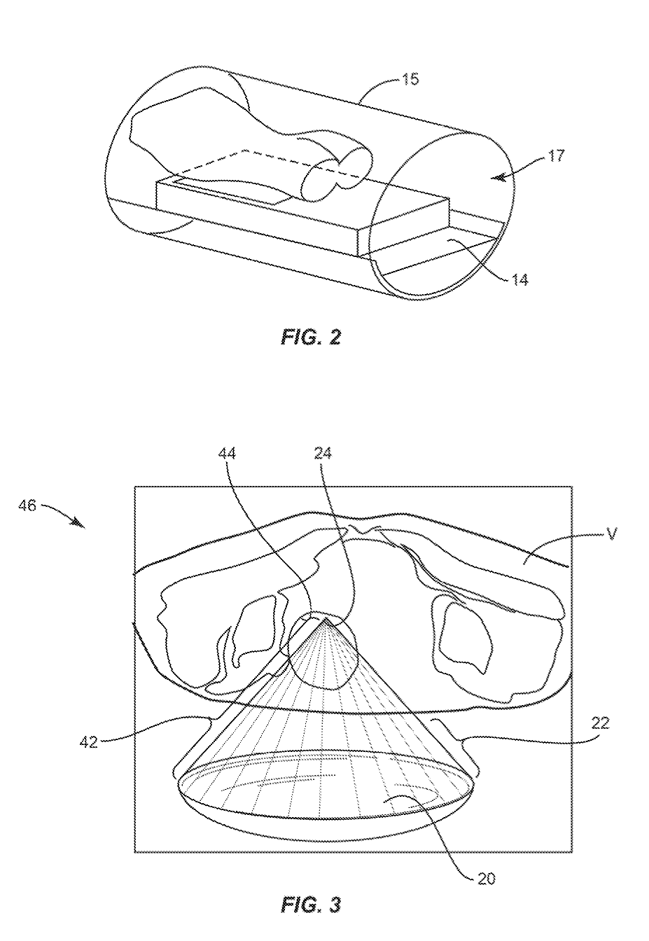

In one embodiment, system 10 includes a positioning system, such as, for example, patient table 14, as shown in FIG. 2, configured for holding and positioning a patient relative to MRI device 12 and HIFU device 16. Table 14 has five degrees of freedom, such as, for example, translation in the X, Y, and Z axes and rotation about the X and Y axes. Patient table 14 is electrically coupled to processor 32 such that processor 32 executes algorithms and/or methods of employment of system 10, as described herein, which direct the movement of table 14 into the desired position to target specific spinal tissue for ablation for targeting of focal point 24.

System 20 includes a computer readable medium 34 for storing at least one algorithm and/or methods of employment of system 10, and may include software programs, applications and codes for determining treatment, such software programs, applications and codes being readily prepared by one skilled in the art based on the present disclosure, to be executed by processor 32 for engaging in the methods of use of system 10. Processor 32 executes the at least one algorithm. System 20 includes a user interface 33. User interface 33 includes a monitor 36 for providing a visual representation of the positioning of focal point 24 and the target tissue site. User interface 33 further includes a computer terminal 30 and a workstation 38. A user or practitioner can input information into user interface 33, such as, for example, the at least one algorithm. User interface 33 is coupled to HIFU device 16 and MRI device 12.

System 10 includes a heat sensor 40 configured to monitor the temperature of healthy portions of the spine adjacent to the portion of the spine being targeted by focal point 24 and requiring treatment. Heat sensor 40 provides the temperature of surrounding and/or adjacent tissue such that a practitioner can prevent damage to healthy tissue by HIFU device 16. If healthy tissue exceeds a certain threshold temperature, such as, for example, over 70° C., HIFU device 16 may be turned off, pulsed, repositioned, and/or have the intensity adjusted to prevent coagulation necrosis of healthy tissue.

The at least one algorithm and/or method of use of system 10 accounts for the complex anatomy of the spine such that transducer 18 can be positioned and focal point 24 focused relative to the portion in the spine requiring treatment. The complex bony structure of the spine provides obstructions 21 angled in disorderly and ill-defined orientations such that ultrasonic beams 22 are refracted along an acoustic path 42, shifting and altering focal point 24. The algorithms and/or method of use of system 10 take into account these obstructions 21 to minimize their impact on the trajectory of ultrasonic beams 22 so that the portion in the spine requiring treatment is the portion in the spine that receives a HIFU beam.

Algorithms and/or methods of use of system 10 also account for the unique biochemical composition of the anatomical structures in the spine, such as, for example, discs and nerves. In some embodiments, algorithms relevant for formulating a HIFU dosage for treating the spine, such as, for example, discs and nerves include, but are not limited to transducer 18 having a focal length from about 25 μm to about 3500 μm, a focal distance from about 80 mm to about 200 mm, a diameter from about 80 mm to about 300 mm, a working frequency of about 0.5 MHz to about 1 GHz and an amplitude from about 1 micron to about 100 microns.

In some embodiments, the focal length is about 3500 μm, 3250 μm, 3000 μm, 2750 μm, 2500 μm, 2250 μm, 2000 μm, 1750 μm, 1500 μm, 1250 μm, 1000, μm 750 μm, 500 μm, 250 μm, about 175 μm, 150 μm, 125 μm, 100 μm, 75 μm, 50 μm and 25 pm for treating the anatomical structures in the spine. In some embodiments, the focal length is about 140 μm, 70 μm, 50 μm and 35 μm for treating the anatomical structures in the spine. In some embodiments, the focal distance is about 80 mm, 90 mm, 100 mm, 110 mm, 120 mm, 130 mm, 140 mm, 150 mm, 160 mm, 170 mm, 180 mm, 190 mm and 200 mm for treating the anatomical structures in the spine. In some embodiments, the diameter is about 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, 290 to about 300 mm for treating the anatomical structures in the spine. In some embodiments, the working frequency range for transducer 18 is from about 20 kHz to about 100 kHz, from about 25 kHz to about 50 kHz, or from about 30 kHz to about 50 kHz for treating the anatomical structures in the spine. In some embodiments, the working frequency range for transducer 18 is from about 0.7 MHz to about 3 MHz, from about 0.5 MHz to about 1.0 MHz, from about 0.7 MHz to about 1.0 MHz, from about 1.0 MHz to about 10.0 MHz, or from about 5.0 MHz to about 10.0 MHz for treating the anatomical structures in the spine. In some embodiments, the working frequency of transducer 18 is from about 0.5 MHz to about 2 MHz, from about 100 MHz to about 400 MHz and from about 500 MHz to about 1 GHz for treating the anatomical structures in the spine. In some embodiments, the amplitude is from about 1 micron to about 5 microns, from about 5 microns to about 10 microns, from about 5 microns to about 30 microns, from about 30 microns to about 100 microns for treating the anatomical structures in the spine. In one embodiment, the algorithm may include the type of tissue being treated, the age of the patient, the condition of the spine and the size of a spinal defect or location.

In one example, system 10 is employed to treat a herniated nucleus pulposus (HNP). A spinal disc is composed of an outer annular fibrosis composed of type I collagen and an inner nucleus pulposus consisting primarily of type II collagen, hyaluronan long chains and proteoglycan. The hyaluronan long chains have regions with highly hydrophilic, side chains. These negatively charged regions hydrate the nucleus of the disc by osmosis. The major proteoglycan constituent is aggrecan, which is connected by a link protein to the long hyaluronan. A fibril network, including a number of collagen types along with fibronectin, decorin, and lumican, contains the nucleus pulposus.

The nucleus pulposus disposed within the annulus fibrosis acts as a shock absorber to cushion the spinal column from forces that are applied to the musculoskeletal system. Each vertebra of the spinal column has an anterior centrum or body. The centra are stacked in a weight-bearing column and are supported by the intervertebral discs. A corresponding posterior bony arch encloses and protects the neural elements, and each side of the posterior elements has a facet joint to allow for motion.

The functional segmental unit is the combination of an anterior disc and the 2 posterior facet joints, and it provides protection for the neural elements within the acceptable constraints of clinical stability. The facet joints connect the vertebral bodies on each side of the lamina, forming the posterior arch. These joints are connected at each level by the ligamentum flavum, which is yellow because of the high elastin content and allows significant extensibility and flexibility of the spinal column.

Any disruption of the components holding the spine together (e.g., ligaments, intervertebral discs, facets) decreases the clinical stability of the spine. When the spine loses enough of these components to prevent it from adequately providing the mechanical function of protection, the nucleus pulposus may bulge or herniate from the annular fibrosis resulting in HNP. In some embodiments, algorithms and methods of use of system 10 formulate a HIFU dosage for treating HNP based on the above described spinal anatomy and include, but are not limited to transducer 18 having a focal length from about 25 μm to about 3500 μm, a focal distance from about 80 mm to about 200 mm, a diameter from about 80 mm to about 300 mm, a working frequency of about 0.5 MHz to about 1 GHz and an amplitude from about 1 micron to about 100 microns.

In one embodiment, system 10 is used to treat a spinal tumor via ablation of a spinal tumor. Spinal tumors are located in the spinal cord and require immediate treatment in order to prevent permanent damage to the spinal cord. In treating spinal tumors, care and precaution is taken to prevent damage to the spinal cord and any surrounding healthy structures during the performance of the treatment.

The spinal cord is protected by three layers of tissue, called spinal meninges, which surround the canal. The dura mater is the outermost layer, and it forms a tough protective coating. Located between the dura mater and the surrounding bone of the vertebrae is a space called the epidural space. The epidural space is filled with adipose tissue, and it contains a network of blood vessels. The arachnoid mater is the middle protective layer. The space between the arachnoid and the underlying pia mater is called the subarachnoid space. The subarachnoid space contains cerebrospinal fluid (CSF). The pia mater is the innermost protective layer. It is very delicate and it is tightly associated with the surface of the spinal cord. The cord is stabilized within the dura mater by the connecting denticulate ligaments, which extend from the enveloping pia mater laterally between the dorsal and ventral roots. The dural sac ends at the vertebral level of the second sacral vertebra.

In cross-section, the peripheral region of the cord contains neuronal white matter tracts containing sensory and motor neurons. Internal to this peripheral region is the gray, butterfly-shaped central region made up of nerve cell bodies. This central region surrounds the central canal, which is an anatomic extension of the spaces in the brain known as the ventricles and, like the ventricles, contains cerebrospinal fluid.

The spinal cord has a shape that is compressed dorso-ventrally, giving it an elliptical shape. The cord has grooves in the dorsal and ventral sides. The posterior median sulcus is the groove in the dorsal side, and the anterior median fissure is the groove in the ventral side. In treating spinal tumors, algorithms and methods of use of system 10 are provided that account for these unique structural components of the spinal cord and surrounding tissue. In some embodiments, algorithms for formulating a HIFU dosage for treating spinal tumors based on the above described spinal anatomy and include, but are not limited to transducer 18 having a focal length from about 25 μm to about 3500 μm, a focal distance from about 80 mm to about 200 mm, a diameter from about 80 mm to about 300 mm, a working frequency of about 0.5 MHz to about 1 GHz and an amplitude from about 1 micron to about 100 microns.

In one embodiment, system 10 is used to treat a hypertrophic ligamentum flavum via ablation of the nerves associated with a ligamentum flavum. The ligamenta flava are ligaments which connect the laminae of adjacent vertebrae, all the way from the axis to the first segment of the sacrum (C2 to S1). They are best seen from the interior of the vertebral canal; when looked at from the outer surface they appear short, being overlapped by the laminae. Each ligament consists of two lateral portions which originate one on either side of the roots of the articular processes, and extend backward to the point where the laminae meet to form the spinous process. The posterior margins of the two portions are in contact and to a certain extent united, slight intervals being left for the passage of small vessels. Each ligamentum flavum consists of yellow elastic tissue, the fibers of which, almost perpendicular in direction, are attached to the anterior surface of the lamina above, some distance from its inferior margin, and to the posterior surface and upper margin of the lamina below. In the cervical region the ligaments are thin, but broad and long. The ligaments are thicker in the thoracic region, and thickest in the lumbar region.

The ligamenta flava have a marked elasticity, which serves to preserve the upright posture, and to assist the vertebral column in resuming it after flexion. The elastin prevents buckling of the ligament into the spinal canal during extension, which would cause canal compression. Hypertrophy of this ligament may cause spinal stenosis because it lies in the posterior portion of the vertebral canal. Targeted ablation of the ligamentum flavum nerves can reduce pain associated with a hypertophic ligamentum flavum. Algorithms are used to account for the unique structure and biochemical composition of the ligamenta flava and surrounding spinal anatomy. In some embodiments, algorithms for formulating a HIFU dosage for treating ligamentum flavum based on the above described spinal anatomy and include, but are not limited to transducer 18 having a focal length from about 25 μm to about 3500 μm, a focal distance from about 80 mm to about 200 mm, a diameter from about 80 mm to about 300 mm, a working frequency of about 0.5 MHz to about 1 GHz and an amplitude from about 1 micron to about 100 microns.

In one embodiment, system 10 is used to treat lower back pain by ablating the basivertebral nerves. The basivertebral nerves are present at the posterior midline of all human thoracic and lumbar vertebrae. The basivertebral nerves transmit pain signals produced at vertebral endplates adjacent to degenerated disks. Ablating these nerves results in the reduction in back pain. Algorithms are used to account for the spinal anatomy. In some embodiments, algorithms for formulating a HIFU dosage for treating basivertebral nerves based on the above described spinal anatomy and include, but are not limited to transducer 18 having a focal length from about 25 μm to about 3500 μm, a focal distance from about 80 mm to about 200 mm, a diameter from about 80 mm to about 300 mm, a working frequency of about 0.5 MHz to about 1 GHz and an amplitude from about 1 micron to about 100 microns.

In one embodiment, system 10 is used to perform a facet rhizotomy. Facet joints are small synovial joints located in pairs on the back of the spine between the superior articular process of one vertebra and the inferior articular process of the vertebra directly above it. The biomechanical function of each pair of facet joints is to guide and limit movement of the spinal motion segment. In the lumbar spine, for example, the facet or zygapophysial joints function to protect the motion segment from anterior shear forces, excessive rotation and flexion. Zygapophyseal joints appear to have little influence on the range of side bending (lateral flexion). These functions can be disrupted by degeneration, dislocation, fracture, injury, instability from trauma, osteoarthritis, and surgery. In the thoracic spine, the zygapophysial joints function to restrain the amount of flexion and anterior translation of the corresponding vertebral segment and function to facilitate rotation. Facet rhizotomy ablates and/or temporarily damages the nerves in the facet joints that send pain signals to the brain. Algorithms are used to account for the complex structure of the facet joints and any surrounding tissue. In some embodiments, algorithms for formulating a HIFU dosage for treating a facet rhizotomy based on the above described spinal anatomy and include, but are not limited to transducer 18 having a focal length from about 25 μm to about 3500 μm, a focal distance from about 80 mm to about 200 mm, a diameter from about 80 mm to about 300 mm, a working frequency of about 0.5 MHz to about 1 GHz and an amplitude from about 1 micron to about 100 microns.

In assembly, operation and use, a surgical system, similar to system 10 described above, is employed with a surgical procedure for treatment of a spinal disorder affecting a section of a vertebrae of a patient, as discussed herein. For example, system 10 can be used with a surgical procedure for treatment of a condition or injury of an affected section of the spine including vertebrae V, as shown in FIG. 3.

For example, as shown in FIGS. 1-6, the components of system 10 can be employed with a surgical treatment of an applicable condition or injury of an affected section of a spinal column, such as, for example, a herniated nucleus pulposus. System 10 can be employed to perform ablation of spinal tumors, hypertrophic ligamentum flavum, basivertebral nerve and to perform facet rhizotomy. In some embodiments, the components of system 10 may be employed with one or a plurality of vertebra.

To treat, for example, a herniated nucleus pulposus (HNP) of a selected section of vertebra V, a patient is positioned within bore 17 of MRI device 12 head-first with his/her chest oriented toward the ground. MRI device 12 is activated and the gradient coils are manipulated to alter the magnetic field at a portion of the spine requiring treatment, such as, for example, portion 44 in the nucleus pulposus, as shown in FIG. 3. Monitor 38 displays a three-dimensional image, such as, for example, image 46 shown in FIG. 3, generated by MRI device 12 identifying portion 44. A practitioner examines image 46 and determines a location in the spine that is to receive a HIFU beam.

A specific treatment site in the spine is selected to treat a certain condition or injury of an affected section of the spine. The specific treatment site will be selected based on one or a plurality of parameters for formulating a HIFU dosage for treating the anatomical structures of the spine. The parameters can include the spinal anatomy/bone tissue, as described herein, and the portions of the spinal anatomy targeted, as described herein, to achieve the desired therapeutic effect. The parameters can also include interaction of HIFU with the specific portion of the bony tissue being targeted, including the relevant dosage of HIFU required to achieve the desired therapeutic effect. The parameters can include elements relating to the HIFU device, such as, for example, focal intensity, focal length, focal distance, working frequency, amplitude, acoustic output, acoustic intensity, scanning rate, temperature and/or acoustic power. Once the specific treatment site has been identified, the appropriate dosage is selected based on the parameters to achieve the desired therapeutic effect.

In some embodiments, the HIFU dosage can be based on parameters including applying energy to tissue, which may include the intensity I (W/cm²) multiplied by the duration t (s) of the exposure (Dose=I×t) in units of J/cm² for treating the anatomical structures of the spine. In some embodiments, a mean dose can be applied to the bony tissue being targeted for treating the anatomical structures in the spine. In some embodiments, the mean dose is about 49,300-62,900 J/cm² for treating the anatomical structures in the spine.

Once the treatment site and the dosage have been selected, HIFU transducer 18 is positioned adjacent to the treatment site such that focal point 24 of HIFU device 16 is in alignment with the desired location in portion 44 of the spine. HIFU transducer 18 can be positioned externally of the patient, or inside the body cavity of the patient. Either position will facilitate a non-invasive procedure. In one embodiment, HIFU transducer 18 can be invasively disposed adjacent to the treatment site within the body. In one embodiment, the HIFU transducers having a fixed focal length. In some embodiments, the HIFU transducer has a focal length of 35 mm. In another embodiment, HIFU transducer 18 will include an array of HIFU transducers or elements, enabling variable focal lengths to be achieved.

The at least one algorithm and/or method of use of system 10 accounts for the complex anatomy of the spine and the biochemical composition of the nucleus pulposus, as described herein, and thus positions HIFU transducer 18.

The algorithm and/or method of use of system 10, which is stored in computer readable medium 34, is executed by processor 32 causing transducer 18 to move in at least one of the sagittal and transverse planes. In one embodiment, execution of the algorithm causes patient table 14 to translate and/or rotate relative to transducer 18 within MRI device 12. The execution of the algorithm also sets the intensity and working frequency of transducer 18, as described herein, to account for the biochemical composition of the anatomical structures of the spine.

The accuracy of the position of HIFU device 16 is tested and verified by activating HIFU device 16 to emit a low power beam that is detected by MRI device 12. After the correct trajectory of focal point 24 has been verified, a HIFU beam is applied to the location in portion 44 of the spine for an interval of time, as described herein, to apply the selected HIFU dosage. A plurality of HIFU beams 22 are emitted from concave surface 20 of transducer 18 such that HIFU beams 22 converge at focal point 24 in portion 44 of vertebra V. In one embodiment, HIFU beams are emitted from a pre-selected plurality of channels of transducer 18 such that the HIFU beams travel along an acoustic path free of obstructions, such as, for example, the spinous process and/or pedicle. Applying a HIFU beam to the location in the portion of the spine requiring treatment includes heating the location until coagulation necrosis occurs.

The HIFU beam can be applied in the form of a pulse to protect the neural structures of the spine from heat profusion. An algorithm, as described herein, can be configured to pulse the HIFU beam at a particular interval preventing surrounding healthy tissue from exceeding a threshold temperature. The temperatures of healthy portions of the spine adjacent to the portion of the spine receiving treatment are monitored using heat sensor 40. The temperature of portion 44 of the spine can be monitored by viewing the temperature images generated by MRI device 12 in real-time on monitor 38. Transducer 18 and/or table 14 is repositioned a plurality of times until focal point 24 contacts and ablates all of portion 44 in the spine such that the spinal pathology is treated. The patient is then removed from MRI device 12.

In one embodiment, system 10 is used to treat a spinal tumor via ablation of the spinal tumor via application of a HIFU dosage to the spinal tumor. HIFU transducer 18 is positioned adjacent to the treatment site such that focal point 24 of HIFU device 16 is in alignment with the spinal tumor. Positioning HIFU transducer 18 includes computing at least one algorithm, as described herein, that accounts for the complex anatomy of the spinal column and the spinal cord. A HIFU dosage is formulated based on one or a plurality of parameters, as described herein. After the correct trajectory of focal point 24 has been verified, a HIFU beam is applied to the location of the spine for an interval of time, as described herein, to apply the selected HIFU dosage.

In one embodiment, system 10 is used to treat a hypertrophic ligamentum flavum via ablation of the nerves associated with a ligamentum flavum via application of a HIFU dosage to the nerves. HIFU transducer 18 is positioned adjacent to the treatment site such that focal point 24 of HIFU device 16 is in alignment with the nerves associated with the ligamentum flavum. Positioning HIFU transducer 18 includes computing at least one algorithm that accounts for the complex anatomy of the spine including the ligamenta flava. A HIFU dosage is formulated based on one or a plurality of parameters, as described herein. After the correct trajectory of focal point 24 has been verified, a HIFU beam is applied to the location of the spine for an interval of time as described herein, to apply the selected HIFU dosage.

In one embodiment, system 10 is used to treat lower back pain by ablating the basivertebral nerves via application of a HIFU dosage to the nerves. HIFU transducer 18 is positioned adjacent to the treatment site such that focal point 24 of HIFU device 16 is in alignment with the basivertebral nerves. Positioning HIFU transducer 18 includes computing at least one algorithm that accounts for the complex anatomy of the spine and the location of the basivertebral nerves. A HIFU dosage is formulated based on one or a plurality of parameters, as described herein. After the correct trajectory of focal point 24 has been verified, a HIFU beam is applied to the location of the spine for an interval of time, as described herein, to apply the selected HIFU dosage.

In one embodiment, system 10 is used to perform a facet rhizotomy via application of a HIFU dosage. HIFU transducer 18 is positioned adjacent to the treatment site such that focal point 24 of HIFU device 16 is in alignment with nerves associated with the facet joint being treated. Positioning HIFU transducer 18 includes computing at least one algorithm that accounts for the complex anatomy of the spine including the facet joints and associated nerves. A HIFU dosage is formulated based on one or a plurality of parameters, as described herein. After the correct trajectory of focal point 24 has been verified, a HIFU beam is applied to the location of the spine for an interval of time, as described herein, to apply the selected HIFU dosage.

In one embodiment, a method for treating a spine is provided, as shown in FIG. 6. The method comprises the steps of providing a magnetic resonance imaging (MRI) device 148; identifying with the MRI device a portion of the spine including a surgical site for treatment of a disorder 150; providing a high intensity focused ultrasound (HIFU) device including a probe for emitting ultrasound energy 152; determining parameters of treatment for the surgical site 154; and applying a dosage of ultrasound energy to the surgical site with the HIFU device for treating the disorder 156.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for treating a spine, the method comprising the steps of:
providing a magnetic resonance imaging (MRI) device;
identifying a surgical site for treatment of a spinal disorder with the MRI device, the surgical site including a portion of a spine;
providing a high intensity focused ultrasound (HIFU) device including a transducer for emitting ultrasound energy;
determining parameters of treatment for the surgical site; and
applying a dosage of ultrasound energy to the surgical site with the HIFU device for treating the disorder.

2. The method according to claim 1, further comprising the step of providing a computer storage medium that includes at least one algorithm comprising the dosage and a processor that executes the at least one algorithm.

3. A method as recited in claim 1, wherein the transducer emits a HIFU beam and a focal point of the HIFU beam is within 0.5 mm of the portion of the spine requiring treatment.

4. A method as recited in claim 3, wherein the transducer has a focal distance from 80 mm to 200 mm, a diameter from 80 mm to 300 mm and a working frequency of 0.5 MHz to 2 MHz.

5. A method as recited in claim 1, wherein the portion of the spine includes at least one of a herniated nucleus pulposus and spinal tumors.

6. A method as recited in claim 1, wherein the portion of the spine includes at east one of a hypertrophic ligamentum flavum, a basivertebral nerve and a facet.

7. A method as recited in claim 1, wherein the transducer applies a HIFU beam to a location in the portion of the spine requiring treatment in the form of a pulse to protect the neural structures of the spine from heat profusion.

8. A method as recited in claim 1, further comprising the step of providing at least one algorithm comprising the dosage that is configured to account for a biochemical composition of a disc and/or nerves of the spine.

9. A method as recited in claim 1, further comprising monitoring a temperature of healthy portions of the spine adjacent to the portion of the spine requiring treatment using a heat sensor.

10. A method as recited in claim 1, wherein the step of applying the HIFU beam to the location in the portion of the spine requiring treatment includes heating the location until coagulation necrosis occurs.

11. A method as recited in claim 1, wherein the HIFU device and the MRI device are coupled to a monitor, a computer terminal and a workstation to provide information to a user and for a user to input information.

12. A magnetic resonance-guided high intensity focused ultrasound system for treating a spine, the system comprising:
a MRI device for identifying a portion in the spine requiring treatment; and
a HIFU device including an ultrasound transducer configured to treat the portion in the spine requiring treatment.

13. A system as recited in claim 12, further comprising a computer readable medium storing at least one algorithm and a processor that executes the at least one algorithm.

14. A system as recited in claim 13, wherein the at least one algorithm accounts for a complex anatomy of the spine such that a focal point of a HIFU beam discharged from the HIFU device is within 0.5 mm of the portion in the spine requiring treatment.

15. A system as recited in claim 12, wherein the MRI device is adapted to obtain three-dimensional images in real-time.

16. A system as recited in claim 12, wherein the transducer includes a focal distance from 80 mm to 200 mm, a diameter from 80 mm to 300 mm and a working frequency of 0.5 MHz to 2 MHz.

17. A system as recited in claim 12, wherein the at least one algorithm is configured to account for the biochemical composition of the disc and/or nerves of the spine.

18. A system as recited in claim 12, further comprising a heat sensor to monitor the temperature of healthy portions of the spine adjacent to the portion of the spine requiring treatment.

19. A system as recited in claim 12, further comprising a monitor, a computer terminal and a workstation to provide information is a user and for a user to input information into the system, the monitor, computer and workstation being coupled to the MRI device and the HIFU device.

20. A method for treating a spine, the method comprising the steps of:
providing a magnetic resonance imaging (MRI) device;
identifying a surgical site for treatment of a spinal disorder with the MRI device, the surgical site including a configuration and/or biochemical composition of a selected anatomy of a spine;
providing a high intensity focused ultrasound (HIFU) device including a transducer for emitting ultrasound energy;
determining parameters for treatment of the selected anatomy based on the configuration and/or biochemical composition of the selected anatomy;
formulating a dosage of ultrasound energy based on the parameters; and
applying a dosage of ultrasound energy to the selected anatomy with the HIFU device for treating the disorder.
